(19) United States
(12) Patent Application Publication
(10) Pub. No.: US 2009/0270700 A1
(43) Pub. Date: Oct. 29, 2009

(54) NON-INVASIVE GLUCOSE SENSOR

(75) Inventors: Maarten Marinus Van Herpen, Eindhoven (NL); Olaf Such, Eindhoven (NL); Golo von Basum, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/297,235

(22) PCT Filed: Apr. 18, 2007
(86) PCT No.: PCT/IB2007/051395

§ 371 Date: Oct. 15, 2008

(30) Foreign Application Priority Data
Apr. 21, 2006 (EP) 06300390.9

(51) Int. Cl. A61B 5/1455; A61B 5/08

(52) U.S. Cl. 600/316; 600/532

(57) ABSTRACT

Apparatus and method for sensing HO activity, and in particular blood glucose level based on an analyte level determination, the analyte being carboxyhemoglobin. In a preferred embodiment, HO activity and/or blood glucose level are extrapolated from Hb-CO level by determining an intermediate CO level. The apparatus and method are preferably non invasive.

The present invention relates to an apparatus and method for determining an analyte level in blood, and is more particularly related to an apparatus and method for non-invasively determining the glucose level in blood.

Diabetes is a disease related to a failure of the biological mechanisms of regulation of the glycemia, i.e. the concentration of glucose in blood. In order to help regulate the glycemia during the day and to reduce the numerous physiological problems that can occur to patients suffering diabetes—among others complicated degenerative affections which, in the eye, are especially retinopathy, metabolic affections of the uvea or cataracts—blood glucose level must be monitored as often as possible. This monitoring is essential to help determining when insulin needs to be injected, and in which quantity. Non invasive glucose sensors are therefore highly desirable, to increase the frequency of proper monitoring for patients, which won't have to use a finger prick several times a day, this operation being painful and a potential source of infections.

Different systems have been proposed to non-invasively monitor blood glucose. The systems generally rely on spectroscopic techniques, typically based on the absorption of glucose in the infrared/mid infrared region, using one or more wavelengths to irradiate a sample tissue, usually a body part such as a finger tip or ear lob, where there are enough blood vessels and not too many skin layers. The reflected and/or transmitted light intensity is collected and analysed, and the glucose level is calculated, based on the absorbance data and the collected spectra. Such a sensor based on near infrared spectroscopy is described in U.S. Pat. No. 4,655,225, wherein blood glucose determination is performed by analysing infrared light transmitted through a finger. The light source has a range from 1000 to 2500 nm, and blood glucose level is determined using two preferred wavelengths.

However, a number of other substances have strong spectroscopic properties at the wavelengths used for sensing glucose. These molecules, such as water, salts or fats, therefore can interfere with the measurement of glucose level, resulting in a poor selectivity due to overlapped spectral bands of all substances. Highly overlapped spectra require the use of multivariate calibration mathematics and substantial numbers of calibration spectra, with associated glucose values to develop models capable of extracting the relevant glucose information buried into other information.

Moreover, the accuracy of current non invasive glucose sensors is typically around 1.6 mmol/L, whereas a preferred accuracy is of the order of 1 mmol/L. Hence, there is a need for improved selectivity and sensitivity for non invasive glucose measurement.

It is therefore an object of the present invention to provide an apparatus and method for non-invasively determining glucose level in blood in a reliable and accurate way.

The present invention discloses an apparatus for determining heme oxygenase activity and/or a blood glucose level, the apparatus comprising: blood generated CO determination means for determining a blood generated CO level, and first extrapolating means for extrapolating said HO activity and/or said blood glucose level according to said blood generated CO level.

The invention proposes to determine HO activity and/or the blood glucose level through a blood CO determination. A proper extrapolation may lead to a value of HO activity, and of blood glucose level.

Carboxyhemoglobin is a stable compound, which results from the interaction of carbon monoxide (CO) with hemoglobin. The affinity of hemoglobin for carbon monoxide is ˜240 times higher than that of oxygen, which means that CO competitively binds to the oxygen-carrying hemoglobin, dissociating oxygen and depriving tissues of their oxygen supply. Thus, CO is a poisonous gas, whose inhalation with strong amounts involves faintnesses, headaches, then asthenia (intense weakness), and finally death by asphyxiation. Hb-CO can dissociate in the lungs, releasing the CO molecules into the exhaled breath. Moreover, CO is also endogenously produced in humans. The main source of CO is enzymatic heme breakdown into biliverdin, which is induced by the enzyme heme oxygenase-1 (HO-1). There are three products of this reaction—bilirubin, CO and ferritin, and the CO that is generated in heme breakdown binds to hemoglobin, forming carboxyhemoglobin. The amount of CO endogenously produced in the body in a given time frame will be referred to as blood generated CO.

Thus, CO in the exhaled breath may include CO that was generated in heme breakdown, as well as CO coming from Hb-CO dissociation in the lungs.

Therefore, the amount of CO measured in the breath is linked directly to the blood Hb-CO level, which, in turn, is a measure of the amount of CO which has been absorbed into the blood stream. Because blood CO comprise blood generated CO, and since heme breakdown is induced by the heme oxygenase, the amount of blood CO can be linked to blood Hb-CO and to HO activity.

Besides, the effects of diabetes on the level of exhaled CO have been studied (Paredi P, Biernacki W, Invernizzi G, Kharitonov S A, Barnes P, “Exhaled carbon monoxide levels elevated in diabetes and correlated with glucose concentration in blood”, Chest 116 (4), 1007-1011 (1999)). The level of exhaled CO in the breath was found to be higher in patients suffering diabetes, and a correlation could be made between exhaled CO and blood glucose level. In an oral glucose tolerance test (OGTT), an increase in blood glucose level (from 3.9 to 5.5 mmol/L) was associated with an increase in exhaled CO (from 3.0 to 6.3 ppm).

This relation may be explained by different factors, such as the activation of the enzyme heme oxygenase by glucose (R. Henningsson, P. Alm, P. Ekstroem, I. Lundquist, “Heme Oxygenase and Carbon Monoxide: Regulatory Roles in Islet Hormone Release”, Diabetes 48, 66-77 (1999)), and the positive modulation of CO on insulin secretion, whereby acute CO level increases may be part of a counter-regulatory mechanism activated in response to changes in glucose levels. It may also be a reflection of HO activation in response to the oxidative stress induced by hyperglycemia. Hence, the activation of the HO enzyme results in an increase of CO produced by heme breakdown, and thus in an increase of exhaled CO.

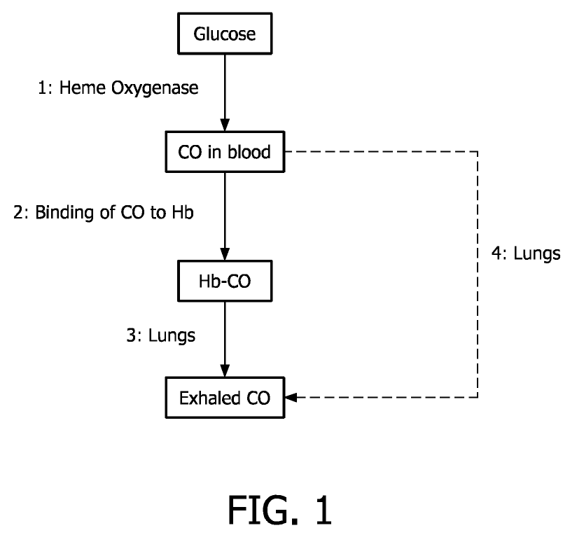

In summary, the relation between heme oxygenase and CO can be explained by the (simplified) model that is shown in FIG. 1.

First, for example, there is a small change in the blood glucose value. This activates the enzyme heme oxygenase (HO) (arrow 1 in FIG. 1), which breaks down heme into bilirubin, CO and ferritin.

The CO molecules that are formed due to this will quickly bind to hemoglobin (Hb) to form Hb-CO (arrow 2 in FIG. 1). The affinity of hemoglobin for CO is ˜240 times that for oxygen, which makes this process very fast. It is expected that all CO will quickly bind to Hb-CO, after which the CO is slowly released and exhaled when Hb-CO dissociates in the lungs (arrow 3 in FIG. 1).

However, it may also be possible that some of the CO can escape through the lungs, before it binds to Hb-CO (arrow 4 in FIG. 1).

Therefore, a first correlation between blood Hb-CO level and CO level in exhaled breath is known from CO poisoning studies, while diabetes studies have proven that a second correlation exists between said CO level in exhaled breath and blood glucose level. The invention suggests to take advantage of the two aforementioned relations, and, this combination leads to a new extrapolation of HO activity, from said blood Hb-CO level and breath CO level. HO presents anti-inflammatory, antiapoptotic, and antiproliferative functions, and its beneficial effects have now been described in diseases as diverse as atherosclerosis and pre-eclampsia: monitoring HO activity is a way to help understanding the mechanisms by which this enzyme gives its protection.

Moreover, this combination also leads to an advantageous determination of blood glucose level that permits a continuous monitoring. Blood glucose level must be monitored as often as possible, in order to help regulate the glycemia during the day and to reduce the numerous physiological problems that can occur to patients suffering diabetes—among others complicated degenerative affections which, in the eye, are especially retinopathy, metabolic affections of the uvea or cataracts. This monitoring is essential to help determining when insulin needs to be injected, and in which quantity.

In an exemplary embodiment of the invention, HO activity and/or blood glucose level can be extrapolated from blood generated CO level, where the assumption is made that only the CO that has been generated in the body in a given time frame and that has escaped in the lungs can be linked to glucose, while the CO coming from the dissociation of Hb-CO represents the background signal in the measurement.

Indeed, Hb-CO has a very slow half-life time, which means that the CO will only slowly be released in the exhaled breath. Hb-CO may be representative of the general environment, such as a city or a countryside where the levels of ambient CO, thus of blood CO, are different, as well as an average of any CO changing effects.

Hence, by providing blood generated CO determination means and extrapolating means, HO activity and glucose level can be determined. In this approach, it is assumed that all or always the same fraction of CO coming from heme breakdown is released in the lungs directly, without binding to haemoglobin, and can be linked to the activity of heme oxygenase and to glucose.

According to a specific aspect of the invention, said blood generated CO determination means comprise breath CO sensing means, for sensing a breath CO level in breath, Hb-CO sensing means, for sensing a blood carboxyhemoglobin (Hb-CO) level, and blood generated CO level extrapolating means, for extrapolating said blood generated CO level according to said Hb-CO level and said exhaled CO level.

Indeed, total breath CO comprises said blood generated CO and said CO that was bound to hemoglobin, forming Hb-CO. Thus, by providing breath CO sensing means, a breath CO level in breath can be determined. Similarly, by providing Hb-CO sensing means, blood carboxyhemoglobin (Hb-CO) level can be determined. Said blood generated CO can then be extrapolated as a function of Hb-CO level and of breath level.

Accordingly, said blood generated CO level extrapolating means may further comprise CO computing means for computing a computed CO level according to said blood Hb-CO level. A relation converting the amount of CO measured in the exhaled breath to the blood Hb-CO level (“Carboxyhemoglobin Levels”, http://www.indsci.com/docs/Gas_Carboxy_Intrinsic.pdf) is well known, in particular when studying CO poisoning. By providing computing means adapted for computing a CO level, a CO level can be deducted from a sensed blood Hb-CO level. Said CO level extrapolated from blood Hb-CO will be referred to as computed CO level.

For example said breath CO sensing means is adapted to measure both inhaled CO level and exhaled CO level. Indeed, the amount of exhaled CO depends on the amount of Hb-CO, as described before, and on the amount of blood generated CO. Additionally, also the CO that has been inhaled, may at least partially be exhaled again. Hence, by providing a CO sensing means adapted to measure both inhaled CO level and exhaled CO level, all contributions to CO exhaled level can then be taken into account, and the measurement may be more accurate.

In an embodiment, said breath CO sensing means is an optical gas sensor. The sensor may use for example direct absorption spectroscopy, photo-acoustic spectroscopy, cavity ringdown spectroscopy, or cavity leak-out spectroscopy

Accordingly, said blood generated CO level is a function of said exhaled CO level, said inhaled CO level, and said computed CO level.

The present invention also discloses an apparatus for determining heme oxygenase activity and/or a blood glucose level, the apparatus comprising Hb-CO sensing means for sensing a blood carboxyhemoglobin (Hb-CO) level, and second extrapolating means for extrapolating said HO activity and/or said blood glucose level according to said Hb-CO level.

The determination of the blood glucose level and/or HO activity may be performed through an analyte level determination, the analyte being carboxyhemoglobin (Hb-CO). A proper extrapolation may lead to a value of blood glucose level and/or HO activity.

Referring again to FIG. 1, the relation between heme oxygenase and carbon monoxide is explained. A small change in blood glucose level activates heme oxygenase, tus inducing heme breakdown. CO molecules that are formed will partly bind to haemoglobin, forming Hb-CO, which breakdowns in the lungs releasing CO. The correlation between blood Hb-CO level and CO level in exhaled breath is known from CO poisoning studies, while diabetes studies have proven that a second correlation exists between said CO level in exhaled breath and blood glucose level. The second preferred embodiment of the invention suggests to take advantage of these relations, and, in a very advantageous way, this combination leads to a new extrapolation of HO activity and blood glucose level from said blood Hb-CO level.

Thus, by providing an apparatus comprising Hb-CO sensing means and glucose level extrapolating means, blood Hb-CO level can be sensed in a first step, leading to a blood glucose level and/or HO activity determination on the basis of said sensed blood Hb-CO level.

In an embodiment, said second extrapolating means comprises CO level computing means for computing a carbon monoxide level according to said Hb-CO level. Moreover, said second extrapolating means further comprises glucose level computing means for computing said blood glucose level according to said computed CO level.

By providing a first extrapolation between exhaled CO and Hb-CO, CO level can be computed by said CO level computing means, and, in a similar way, glucose level computing means can compute said blood glucose level, using the extrapolation between exhaled CO and blood glucose level.

Moreover, an apparatus according to the invention may comprise CO level calibration means for including an ambient CO level in the computation of said glucose level according to said CO level.

Indeed, depending on the general environment, the ambient CO level may vary, thus influencing the blood CO level. For instance, the blood CO level may be higher in a city than in the countryside, or may depend on the degree of pollution, and on other external factors. Since the extrapolation of blood glucose level relies on a single extrapolation of a CO level from Hb-CO level, said extrapolated blood glucose level may be shifted, depending on said ambient CO level. Therefore, it would be necessary to take account of an ambient CO level, to ensure that the extrapolation of blood glucose level leads to a correct value.

In an exemplary embodiment, said CO level calibration means may comprise CO sensing means for sensing an ambient CO level, and modelling means for modelling Hb-CO level responsive to said ambient CO level. Thus, by providing modelling means for calculating the changes in Hb-CO levels as a result of changes in ambient CO level, and possibly on other parameters, such as heart rate, respiration rate, and others, the accuracy of the glucose level determination may be improved, or the time between calibrations may be increased.

Most preferably, said Hb-CO sensing means is non invasive. A number of invasive and non-invasive hemoglobin sensors (including hemoglobin compounds such as carboxyhemoglobin, oxyhemoglobin, deoxyhemoglobin) have been developed, in particular to detect CO poisoning. By using preferably non invasive Hb-CO sensing means, it becomes possible to provide for a non-invasive blood glucose sensing apparatus, needed to sense blood glucose level a great number of times during the day, without having to endure the inconveniences of an invasive glucose detector.

An apparatus according to the invention may further comprise calibration means for calibrating said HO activity and/or said blood glucose level obtained by extrapolation.

Indeed, when HO activity, and/or blood glucose level, is determined according to a direct measurement of Hb-CO, the measurement may be influenced by different parameters, as blood Hb-CO level may be influenced by a great number of parameters and physiologic functions, among others asthma, hypertension, sepsis or pre-eclampsia (D. Morse, A. M. K. Choi, “Heme Oxygenase-1. The ‘Emerging Molecule’ Has Arrived”, Am. J. Respir. Cell Mol. Biol. 27, 8-16 (2002)). The extrapolated glucose level may be shifted, depending on the current Hb-CO level. Therefore, an apparatus according to the invention preferably comprises glucose level calibration means, in order to calibrate the relation between Hb-CO and glucose, and HO activity.

When blood glucose level is determined according to blood generated CO level, different types of carbon monoxide are sensed and computed, thus potentially leading to deviations from the real blood glucose level during the extrapolation. Hence, and as it is often the case when different extrapolations are used, a calibration may be necessary to improve the accuracy of blood glucose level measurement. Because HO activity may be linked directly to the amount of said blood generated CO, a calibration may indeed be required.

Said calibration means may comprise at least one of reference glucose sensing means for sensing a reference glucose level, and/or comparison means for comparing values of said blood glucose level and/or said HO activity obtained at different measurement times

Calibrations of different kinds are contemplated. One option would be to sense a reference glucose level using reference glucose sensing means. In this case, the calibration means may be adapted to sense a reference glucose level and to adjust said extrapolated glucose level to said reference glucose level.

Another option could rely on comparison means, for comparing values of said blood glucose level and/or said HO activity obtained at different measurement times.

Depending on the user's needs and on what is detected, heme oxygenase activity and/or blood glucose level, the calibration means may comprise either one or both of the aforementioned calibration options.

Said glucose level calibration means can be adapted to sense a reference glucose level and to adjust said extrapolated glucose level to said reference glucose level, on a regular time basis, possibly on a daily basis.

Indeed, and particularly when blood glucose level is determined directly according to a sensed blood Hb-CO, different parameters may influence the measurement, as blood Hb-CO level may be influenced by a great number of parameters and physiologic functions. These other physiologic functions affecting blood Hb-CO level typically give a slow change of Hb-CO level over time. Therefore, after a certain amount of time, it is contemplated to recalibrate the glucose measurement. This calibration would preferably be made on a regular time basis, for example once a day.

On the other hand, when blood glucose level is determined according to blood generated CO level, a calibration may also be necessary, since the assumption is made that always the same fraction of generated CO will escape directly through the lungs. It could be that this fraction vary, depending for instance on the general environment, where the ambient CO influences the level of blood Hb-CO, and then the level of haemoglobin. It is also to be noted that in this case however, blood Hb-CO is considered as a background measure for blood CO.

In an embodiment, said reference glucose sensing means is a fingerstick type glucose sensor, showing the well-defined accuracy and performances needed for reference.

In an embodiment, said comparison means is adapted to make at least one of the following comparisons: values of said HO activity, ratios of said blood generated CO to said breath CO, ratios of said blood generated CO to said computed CO, said values and ratios being obtained at different measurement times.

Indeed, in order to monitor potential disease such as diabetes, comparison means may be useful, as a tool to monitor the evolution of different parameters over time, with a reference measurement, that could be made with another sensor or with the same sensor at different times.

Said Hb-CO sensing means can be adapted to provide a sensing accuracy of at least 0.5% Hb-CO. Indeed, an analysis of the above-mentioned extrapolation data shows that a change of 1.6 mmol/L blood glucose level corresponds to a change of 3.3 ppm exhaled CO, which in turn corresponds with around 0.53% Hb-CO. Therefore a non-invasive Hb-CO detector would need to have accuracy better than roughly 0.5%. Preferred glucose accuracy would be of 1 mmol/L, which corresponds to an Hb-CO accuracy of 0.3%. It is to be noted that an exact absolute value of blood Hb-CO level need not be measured, because the blood glucose determination is calibrated, but instead the repeatability of the measurement is of great importance.

Many different non-invasive carboxyhemoglobin sensors could be used, based on absorption and on spectroscopic properties of Hb-CO.

Accordingly, in one embodiment of the invention, said Hb-CO sensing means may comprise: illuminating means for illuminating a sample tissue with a plurality of wavelengths, collecting means for collecting transmitted and/or reflected light, and Hb-CO computing means for computing the Hb-CO level responsive to transmitted, emitted and/or reflected light intensity.

A technique similar to oximetry based on absorbance data is contemplated. Oximetry is a technique used to measure hemoglobin level, which measures the increased absorbance present during a pulsatile flow when the arterial bed expands from the increased systolic volume. Normally, an oximeter measures the transmission of light at different wavelengths through a sample tissue. Since the different hemoglobin compounds (oxygenated hemoglobin, reduced hemoglobin and carboxyhemoglobin) don't have the same absorption spectra, their concentrations can be determined from the relative absorptions at different wavelengths.

Alternately, said Hb-CO sensing means may comprise illuminating means for illuminating a sample tissue with a plurality of wavelengths, collecting means for collecting transmitted and/or reflected light, spectroscopic means for separating transmitted and/or reflected light depending on spectral bands from said plurality of wavelengths, and Hb-CO computing means for computing said Hb-CO level according to separated transmitted/reflected light intensity.

Accordingly, spectroscopic and interferometric techniques are contemplated, wherein Hb-CO level can be determined from the intensity of the signal light. The sensing techniques may include Raman spectroscopy, photo-acoustic spectroscopy, direct absorption spectroscopy, fluorescence spectroscopy, optical coherence tomography, thermal emission spectroscopy and diffuse reflection spectroscopy. When interferometry is used, like in optical coherence tomography, the light source is split into at least two beams, a reference beam and a probe beam, the probe beam is usually reflected on the sample tissue. After having traveled over different paths, the probe and reference beams are recombined, and interferences can be obtained, with characteristics depending on the sample tissue properties and composition. A preferred apparatus may use a Michelson or a Mach-Zender interferometer, to measure reflection of light from the tissue.

Satisfactory results may be obtained when many different excitation and/or signal wavelengths are used, improving the accuracy of the measurement.

Said plurality of wavelengths is in the range of about 450 nm to about 1900 nm, where carboxyhemoglobin has the strongest absorption. Said plurality of wavelengths may comprise any number of wavelengths, preferably at least three or more for improved reliability and accuracy.

Said illuminating means may comprise light sources, among others light emitting diodes, lasers, halogen sources or any other light sources. Sources of different widths and coherence are contemplated, in particular white light sources, broadband or monochromatic sources. In the latter case, multiplexing means can also be associated, to provide a single illuminating beam on the tissue. Moreover, said illuminating means may further comprise imaging optics, including one or more lenses, light guiding means, reflectors or focusing means, for focusing the light, and direct it into the sample tissue.

Suitable collecting means and spectroscopic means may comprise detectors with a detection window in the range of said plurality of wavelengths, like photodiodes or avalanches photodiodes, integrating spheres, photometers, or any suitable optoelectronics component, wavelength selecting devices, optical spectrum analyzer, spectrometer, whose resolution may be of a few nm over the entire range defined by said plurality of wavelengths. Preferably, detectors have a uniform sensitivity over the range of wavelengths.

Moreover, said collecting means may also include demultiplexing means, for demultiplexing the light at different wavelengths, amplifying means for amplifying the sensed signal, filtering means, with uniform or well-defined response over the wavelengths range.

Computing means comprises filtering, signal processing tools and techniques well-known in the art.

In addition, it is to be noted that free optics or integrated optics, for instance with fiber optics, may be used.

Typically, the sample tissue is a blood profused tissue, such as a finger or an ear lobe. Another tissue of particular interest is the retina, because measuring Hb-CO level in the retina may give an indication of the risk of retina damage due to diabetes, or to screen for diabetes. A retina glucose sensor according to the invention may use reflection spectroscopic techniques. When measuring in the eye, the transparency of the eye must be taken into account. However, there are strong spectroscopic features of Hb-CO within the transparency range of the human eye, i.e., in a range of 400-900 nm.

Accordingly, the invention also proposes a method for determining heme oxygenase activity and/or a blood glucose level, comprising the steps of determining a blood generated CO level, and extrapolating said heme oxygenase activity and/or said glucose level according to said blood generated CO level.

The step of extrapolating said HO activity and/or said glucose level comprises the steps of sensing a breath CO level in breath, sensing a blood carboxyhemoglobin (Hb-CO) level, and extrapolating said HO activity and/or said blood generated CO according to said Hb-CO level and said breath CO level.

In an embodiment, in the step of extrapolating said glucose level and/or said HO activity, the step of sensing a breath CO level comprises sensing an exhaled CO level and an inhaled CO level, the step of extrapolating said blood generated CO level comprises the step of computing a carbon monoxide (CO) level according to said Hb-CO level, and wherein said blood generated CO is a function of said computed CO level, said exhaled CO level, said inhaled CO level.

The invention also provides for a method for non invasively determining HO activity and/or a blood glucose level, comprising the steps of sensing a blood carboxyhemoglobin (Hb-CO) level, and extrapolating said HO activity and/or said glucose level according to said Hb-CO level.

The step of extrapolating said HO activity and/or said glucose level comprises the steps of: computing a carbon monoxide (CO) level according to said Hb-CO level, and computing said glucose level according to said computed CO level.

A method according to the present invention may further comprise the step of calibrating said glucose level and/or said HO activity obtained by extrapolation. The step of sensing blood Hb-CO may be non invasive.

Finally, the invention provides a method for non invasively determining heme oxygenase activity and/or blood glucose level according to anyone of claims 22-24, 27-28 using an apparatus according to anyone of claims 1-6, 12-21, and/or according to anyone of claims 25-28 using an apparatus according to anyone of claims 7-21.

Other features and advantages of the present invention will become apparent from the following description of a preferred embodiment, by way of example only, and with reference to the accompanying drawings, wherein:

FIG. 1 is a diagram explaining the relation between heme oxygenase and carbon monoxide, useful for the present invention,

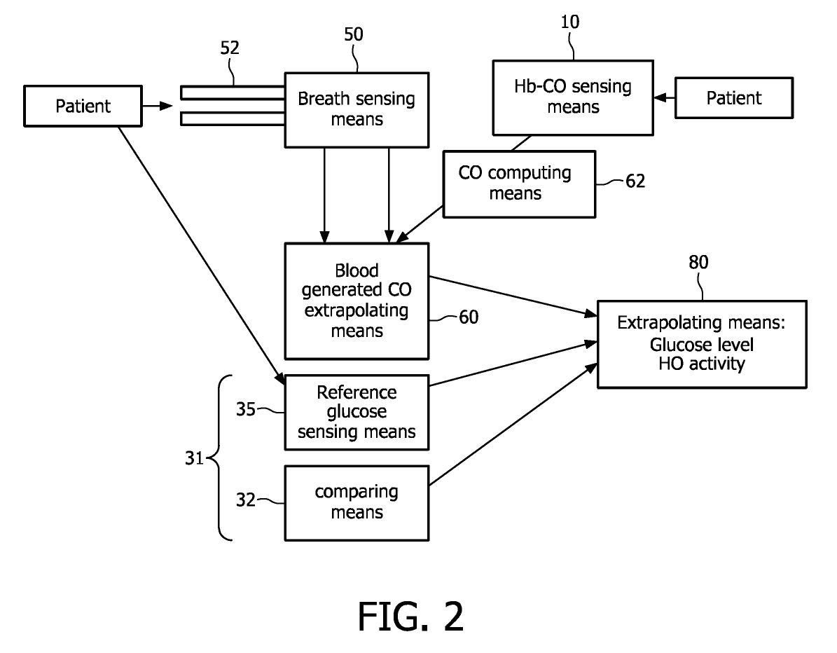

FIG. 2 is a functional diagram of an apparatus for determining HO activity and/or blood glucose level, according to a first preferred embodiment of the present invention,

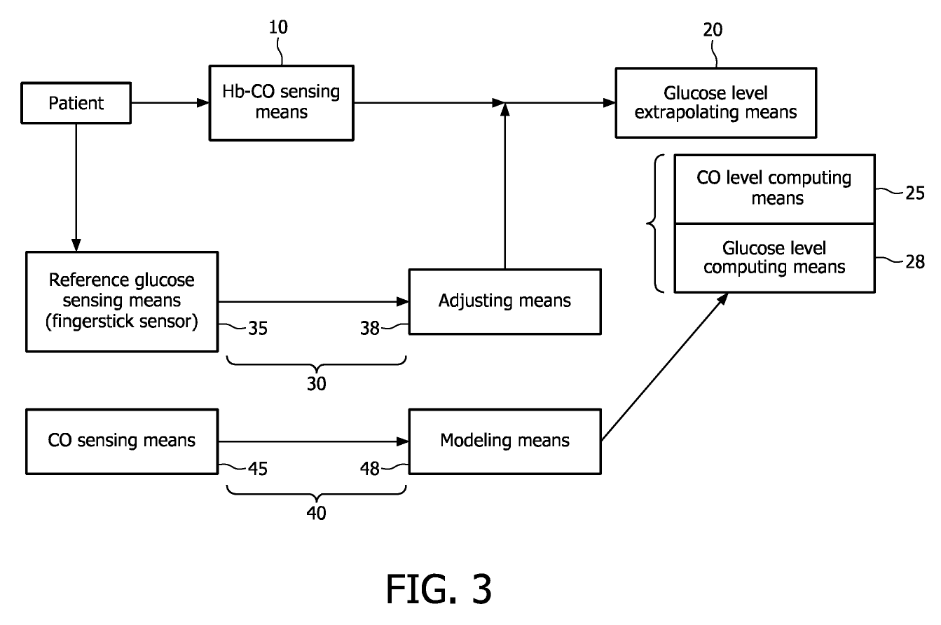

FIG. 3 is a functional diagram of an apparatus for determining HO activity and/or blood glucose level, according to a second preferred embodiment of the present invention,

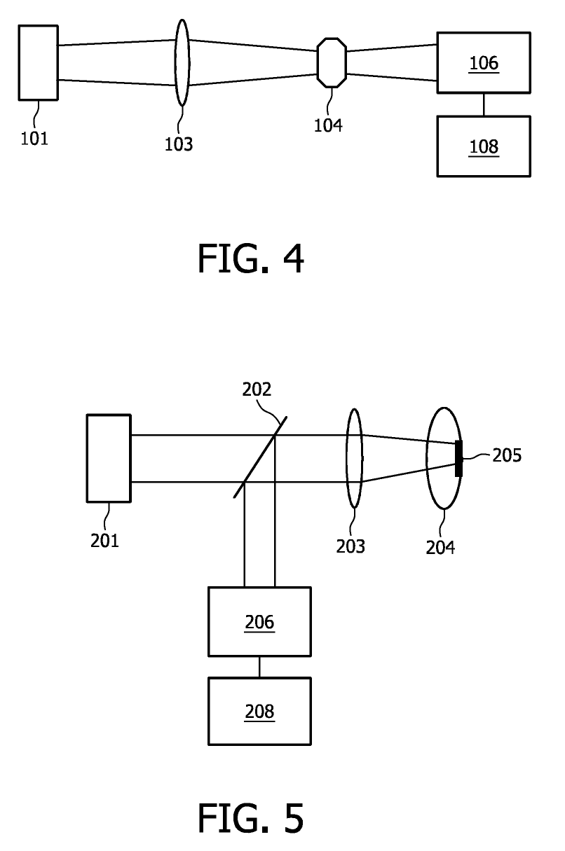

FIG. 4 is a schematic view of a carboxyhemoglobin sensor suitable for use in an apparatus for determining HO activity and/or blood glucose level, according to the present invention,

FIG. 5 is a view of another carboxyhemoglobin sensor in an apparatus for determining HO activity and/or blood glucose level, in an alternative embodiment of the present invention.

In the figures, identical numerical references refer to similar components.

FIG. 2 is a functional diagram of an apparatus for determining HO activity and/or blood glucose level, according to a first preferred embodiment of the present invention.

The apparatus comprises blood generated CO determination means, whereby a blood generated CO is determined, said blood generated CO being defined as the amount of CO produced in a given timeframe (for example by heme breakdown in the patient's body), and extrapolating means 80 for extrapolating HO activity and/or blood glucose level according to said blood generated CO.

Said blood generated CO determination means comprise breath CO sensing means 50, Hb-CO sensing means 10, blood generated CO extrapolating means 60.

Breath CO sensing means 50 may be adapted for measuring both inhaled and exhaled breath of the patient. Preferably, breath CO sensing means 50 are provided with a gas tube 52 through which at least some of the breath of the patient is flown. When the patient inhales, the gas travels away from the breath CO sensing means 50, and when the patient exhales, the gas travels towards the breath CO sensing means 50. In this way, the CO concentration in the inhaled and exhaled breath can be measured.

Breatg CO sensing means 50 may be an optical gas sensor, using for example direct absorption spectroscopy, photo-acoustic spectroscopy, cavity ringdown spectroscopy, or cavity leak-out spectroscopy (CALOS).

The apparatus further comprises a Hb-CO sensing means 10, which are adapted to measure a blood Hb-CO level, and are preferably a non-invasive sensor, using absorption and/or spectroscopic techniques. Preferred embodiments of Hb-CO sensing means will be described in more details with reference to FIGS. 4 and 5.

The data from breath CO sensing means 50 and Hb-CO sensing means 10 are processed by blood generated CO extrapolating means 60. First, CO computing means 62 calculates a CO level according to said Hb-CO level, using the well-known extrapolation between CO and Hb-CO levels. Then, said blood generated CO level is determined using the values of inhaled CO level, exhaled CO level and computed CO level.

From said blood generated CO level, both HO activity and blood glucose level may be calculated.

Of course, said blood generated CO determination means, CO computing means 62, and extrapolating means 60, 80, are also provided with all necessary electronics, signal processing and computing tools.

The HO activity and/or glucose sensing may be performed without harm and as often as necessary, since Hb-CO sensing means 10 are non-invasive. Said extrapolating means 80 preferably comprise display and memory for storing said glucose level data and HO activity data. This feature may be useful for monitoring disease, e.g. to see if the condition of the patient improves or get worse.

The apparatus further comprises calibration means 31. Said calibration means 31 include reference glucose sensing means 35, such as a fingerstick sensor, which is a reliable and accurate glucose sensor. Blood glucose level is measured with the fingerstick sensor 35 on a regular time basis, in particular once a day, as a reference glucose level. This reference glucose level is in turn compared to said extrapolated glucose value. Hence, said calibrations means 31 are provided with electronics, soft and signal processing tools, so as to perform the aforementioned comparison, and to adjust and assign in the extrapolation said extrapolated glucose level to said reference glucose level. This operation may be done once a day, and said calibration means 31 may also include features such as an alarm together with a LED, flashing to warn the user when a calibration is required.

Moreover, said calibration means 31 may also be provided with comparison means 32, in order to make comparisons between different parameters values obtained at different times, as a tool to monitor the evolution of different parameters over time, with a reference measurement, that could be made with another sensor or with the same sensor at different times. Preferably, said comparison means are adapted to make at least one of the following comparisons: values of said HO activity, ratios of said blood generated CO to said breath CO, ratios of said blood generated CO to said computed CO, said values and ratios being obtained at different measurement times.

A possible ratio is the ratio between blood generated CO and breath CO or computed CO, because this value gives the ratio between the currently generated CO and the CO generated in a longer time period. Thus, this ratio can be indicative of the change in CO production and thus the change of HO activity.

Hence, the apparatus of FIG. 2 may be used as follows. When the user starts using the apparatus, a first glucose calibration is performed, consisting in measuring a reference glucose level with said fingerstick sensor, and in determining a first extrapolated glucose level value and/or HO activity value based on blood generated CO measurement. These two values are compared and said extrapolated glucose value is adjusted to said reference value. Later on, when the user needs to perform a new glucose level determination, the user need not perform the glucose level calibration.

Further, in the course of a given time period, for instance a day, every following HO activity and/or glucose determination consists in blood generated CO level determination, based on the sensed values of blood Hb-CO level, exhaled CO and inhaled CO levels, followed by an extrapolation to determine glucose level value and/or HO activity, with all necessary computing steps, and optionally calibration as well.

FIG. 3 is a functional diagram of an apparatus for determining HO activity and/or blood glucose level, according to a second preferred embodiment of the present invention. The apparatus comprises non-invasive Hb-CO sensing means 10, glucose extrapolating means 20, and glucose level calibration means 30.

Hb-CO sensing means 10 are adapted to measure a blood Hb-CO level, and are preferably a non-invasive sensor, using absorption and/or spectroscopic techniques. Preferred embodiments of Hb-CO sensing means will be described in more details with reference to FIGS. 4 and 5.

Glucose level extrapolating means 20 comprises CO level computing means 25 and glucose level computing means 28. Electronics, signal processing and computing tools are provided with CO level computing means 25, which are adapted to compute a value of CO level according to said Hb-CO level, using the well-known relation between blood Hb-CO and exhaled CO. Similarly, said glucose level computing means 28 are also provided with all necessary electronics, signal processing and computing tools, to compute said glucose level, using the aforementioned relation between exhaled CO and blood glucose level.

The glucose sensing may be performed without harm and as often as necessary, since Hb-CO sensing means are non-invasive. Said glucose level extrapolating means 20 preferably comprise display and memory for storing said glucose level data. This feature may be useful for monitoring blood glucose value in time.

The apparatus further comprises glucose level calibration means 30, a calibration being required because Hb-CO level is also influenced by other physiological factors, such as asthma for example, which give typically a slow change of Hb-CO level over time.

Said glucose level calibration means 30 include reference glucose sensing means 35, such as a fingerstick sensor, which is a reliable and accurate glucose sensor, and adjusting means 38. Blood glucose level is measured with the fingerstick sensor 35 on a regular time basis, in particular once a day, as a reference glucose level. This reference glucose level is in turn compared to said extrapolated glucose value obtained through a Hb-CO measurement followed by an extrapolation. Hence, said adjusting means 38 are provided with electronics, soft and signal processing tools, so as to perform the aforementioned comparison, and to adjust and assign in the extrapolation said extrapolated glucose level to said reference glucose level. This operation may be done once a day, and said adjusting means 38 may also include features such as an alarm together with a LED, flashing to warn the user when a calibration is required. It is also contemplated that no Hb-CO sensing is possible as long as the calibration has not been made.

Furthermore, since the ambient CO level may influence the blood CO level and thus the glucose level determination, the apparatus may also be provided with CO level calibration means 40 for including a ambient CO level in the computation of said glucose level according to said CO level. Said CO level calibration means 40 is comprised of CO sensing means 45, such as a gas detector commonly known in the art, together with modelling means 48 for modelling Hb-CO level responsive to said ambient CO level. Modelling means may include a model to evaluate the changes in Hb-CO levels as a result of changes in ambient CO level. Possibly other parameters, such as heart rate, respiration rate, and others, may also be taken into account. Once again, all necessary electronics and signal processing tools are provided with said modelling means 48.

Hence, the apparatus of FIG. 3 may be used as follows. When the user starts using the apparatus, a first glucose calibration is performed, consisting in measuring a reference glucose level with said fingerstick sensor, and in determining a first extrapolated glucose level value based on Hb-CO level measurement. These two values are compared and said extrapolated glucose value is adjusted to said reference value. Later on, when the user needs to perform a new glucose level determination, the user need not perform the glucose level calibration, for a given time period, for example a day. Another calibration may also include a ambient CO level, measured through a simple gas detector, and being included in a model for determining the variations of Hb-CO level responsive to ambient CO variations. This calibration may be included at each glucose level determination.

Further, in the course of a given time period, for instance a day, every following glucose determination consists in blood Hb-CO level determination followed by an extrapolation to determine glucose level value, with all necessary computing steps, and optionnaly the CO calibration as well. When the given time span expires, the user may be warned to perform a new glucose calibration.

Preferred Hb-CO sensing means, for measuring blood Hb-CO and hence blood glucose level in a tissue sample is depicted FIG. 4, said Hb-CO sensing means relying on the same principles as pulse oximetry, with additional wavelengths.

Hb-CO sensing means 10 comprise a light source 101, which is directed towards the tissue bed, for example an ear lob 104. The light source 101 may be a broadband light source, in the range of 400-1900 nm, where carboxyhemoglobin has strong absorption properties, the light source being preferably uniform over the given range.

Preferably, one or more monochromatic sources are used, such as laser diodes, preferably of the same width, with their driving electronics. Different measurements can be performed and added. Another option is to use different monochromatic sources along with a multiplexer and to perform a single measurement.

The light beam from the light source passes through imaging optics 103, comprising one or more lenses for focusing the light. The light beam is then focused onto the ear lob 104. When passing through the ear lob 104, light is absorbed, and, in a first linear approximation, the absorbance is given by the Beer-Lambert law, wherein the absorbance at a given wavelength is given by:

A_{λ,i}=Σᵢeᵢ.cᵢ.lᵢ

where eᵢ is the absorptivity of a component i at wavelength λ, cᵢ is the concentration of component i, and l is the light path-length.

The increased absorbance present during a pulsatile flow when the arterial bed expands from the increased systolic volume, is a measure of the hemoglobin components absorption, in particular blood Hb-CO level may be obtained from the absorbance data.

Preferred wavelengths correspond to peak absorption of different hemoglobin components, for example, 530.6 nm und 583 nm. The overall accuracy may be improved by using several wavelengths. However, it is to be noted that the wavelengths don't have to be at the peaks, as contrast, i.e the difference between the signals when the CO level shifts, is the most important parameter. The wavelengths may be 630 nm, 720 nm and 900 nm, in an exemplary embodiment when only three wavelengths are used.

The transmitted light is directed towards a detector 106. The detector 106 preferably has a uniform sensitivity in the range of wavelengths used for sensing Hb-CO, i.e., 400-1900 nm. After signal processing, Hb-CO level is determined from the detected signal, using computing means 1008 with filtering and signal processing tools well-known in the art.

Alternative Hb-CO sensing means, for measuring blood Hb-CO and hence blood glucose level in a sample tissue is depicted FIG. 5, said Hb-CO sensing means relying on reflection spectroscopy. A preferred region of interest is the retina, because measuring Hb-CO level in the retina may give an indication of the risk of retina damage due to diabetes, or may be used to screen for diabetes.

Hb-CO sensing means 10 comprise a light source 201, which is directed towards the eye 204. When measuring in the eye, the transparency of the eye must be taken into account. However, there are strong spectroscopic features of Hb-CO within the transparency range of the human eye, i.e., in a range of 400-900 nm. Therefore, the light source 101 may be a broadband light source, at least in the range of 400-900 nm, preferably uniform in the given range. The light beam from the light source passes through imaging optics 203, comprising one or more lenses for focusing the light. The light beam is then directed into the eye 204 and focused onto the retina 205. Of course, it is important that the intensity of the light source 201 is low enough to make sure that no damage to the retina 205 is caused.

The light reflected off the retina is redirected towards the light source 201. However, a beam splitter 202 sends the reflected light towards a detector 206. The detector 206 preferably has a uniform sensitivity in the range of wavelengths used for sensing Hb-CO in the retina, i.e., 400-900 nm, and includes a spectrometer for measuring the absorption spectrum of the reflected light, or an optical wavelength analyzer. When necessary, an optoelectronic or electrical amplifier may be added, in order to amplify the detected signal.

Hb-CO level is then determined, through the use of computing means 208, including filters and signal processing tools.

It is to be noted that the light sources 101, 201 may be broadband light sources or rely on a set of monochromatic sources, such as laser diodes, preferably of the same width, with their driving electronics. Different measurements can be performed and added. Another option is to use different monochromatic sources along with a multiplexer and to perform a single measurement, as it is the case for broadband light source. Tunable sources may further be employed. When broadband light source are contemplated, spectrometer means may be used in order to measure the intensity of light at several wavelengths.

Thus, an apparatus and a method for determining HO activity is disclosed, and linked to a blood glucose level, based on the determination of a blood analyte level along with extrapolating means to get the actual HO activity as well as the actual blood glucose level, wherein calibration means are also preferably involved. In a very advantageous way, this apparatus may be implemented in a non-invasive way, with improved accuracy and repeatability.

1. Apparatus for determining a blood glucose level, the apparatus comprising:
blood generated CO determination means for determining a blood generated CO level, and
first extrapolating means (80) for extrapolating said blood glucose level according to said blood generated CO level.

2. Apparatus according to claim 1, said blood generated CO determination means comprising:
breath CO sensing means (50) for sensing an exhaled CO level in breath,
Hb-CO sensing means (10) for sensing a blood carboxyhemoglobin (Hb-CO) level, and
blood generated CO level extrapolating means (60) for extrapolating said blood generated CO level according to said Hb-CO level and said exhaled CO level.

3. Apparatus according to claim 2, said blood generated CO level extrapolating means (60) further comprising CO computing means (62) for computing a computed CO level from said blood Hb-CO level.

4. Apparatus according to claim 2, said breath CO sensing means (62) being adapted to measure both inhaled CO level and exhaled CO level.

5. Apparatus according to claim 4, said blood generated CO level being a function of said exhaled CO level, said inhaled CO level, and said computed CO level.

6. Apparatus for determining a blood glucose level, the apparatus comprising:
Hb-CO sensing means (10) for sensing a blood carboxyhemoglobin (Hb-CO) level, and
second extrapolating means (20) for extrapolating said blood glucose level based on said Hb-CO level.

7. Apparatus according to claim 6, said second extrapolating means comprising:
CO level computing means for computing a carbon monoxide (CO) level according to said Hb-CO level.

8. Apparatus according to claim 7, said second extrapolating means further comprising glucose level computing means for computing said blood glucose level according to said computed CO level.

9. Apparatus according to claim 8, further comprising CO level calibration means for including an ambient CO level in the computation of said glucose level according to said computed CO level.

10. Apparatus according to claim 9, said CO level calibration means comprising
CO sensing means for sensing a ambient CO level, and
modelling means for modelling Hb-CO level responsive to said ambient CO level.

11. Apparatus according to claim 2, said Hb-CO sensing means comprising:
illuminating means for illuminating a part of the body with a plurality of wavelengths,
collecting means for collecting transmitted and/or reflected light, and
Hb-CO computing means for computing the Hb-Co level responsive to transmitted, emitted and/or reflected light intensity.

12. Apparatus according to claim 11, said Hb-CO sensing means for sensing said Hb-CO level further comprising:
illuminating means for illuminating a part of the body with a plurality of wavelengths,
collecting means for collecting transmitted and/or reflected light,
spectroscopic means for separating transmitted and/or reflected light depending on spectral bands from said plurality of wavelengths, and
Hb-CO computing means for computing said Hb-CO level according to separated transmitted/reflected light intensity.

13. Apparatus according to claim 11, said plurality of wavelengths being in the range of about 450 nm to about 950 nm.

14. Apparatus according to claim 1, further comprising calibration means for calibrating said blood glucose level and/or said HO activity obtained by extrapolation, said calibration means comprises reference glucose sensing means for sensing a reference glucose level, and comparison means for comparing values of said blood glucose level obtained at different measurement times

15. Apparatus according to claim 14, said comparison means being adapted to make at least one of the following comparisons: ratios of said blood generated CO to said exhaled CO, ratios of said blood generated CO to said computed CO, said ratios being obtained at different measurement times.

16. Method for non invasively determining a blood glucose level, comprising the steps of:
determining a blood generated CO level, and
extrapolating said glucose level according to said Hb-CO level.

17. Method according to claim 16, the step of extrapolating said glucose level comprising the steps of:
sensing a breath CO level in breath,
sensing a blood carboxyhemoglobin (Hb-CO) level, and
extrapolating said HO activity and/or said blood generated CO according to said Hb-CO level and said breath CO level.

18. Method for non invasively determining a blood glucose level, comprising the steps of:
sensing a blood carboxyhemoglobin (Hb-CO) level, and
extrapolating said glucose level according to said Hb-CO level.

19. Method according to claim 18, the step of extrapolating said glucose level comprising the steps of:
computing a carbon monoxide (CO) level according to said Hb-CO level, and
computing said glucose level according to said computed CO level.
